(19) United States
(12) Patent Application Publication
(10) Pub. No.: US 2018/0185308 A1
(43) Pub. Date: Jul. 5, 2018

(54) COMPOSITIONS AND METHODS FOR TREATING INFLAMMATORY DISEASE OR CONDITIONS

(71) Applicant: Matinas BioPharma Nanotechnologies, Inc., Bedminster, NJ (US)

(72) Inventors: Raphael J. MANNINO, Glen Gardner, NJ (US); Ruying Lu, New Providence, NJ (US)

(21) Appl. No.: 15/735,708

(22) PCT Filed: Jun. 17, 2016
(86) PCT No.: PCT/US16/38084

§ 371 Date: Dec. 12, 2017

Related U.S. Application Data
(60) Provisional application No. 62/181,347, filed on Jun. 18, 2015.
(60) Provisional application No. 62/289,025, filed on Jan. 29, 2016.
(60) Provisional application No. 62/347,014, filed on Jun. 7, 2016.

(51) Int. Cl. A61K 31/192; A61K 9/127; A61K 9/107

(52) U.S. Cl. CPC A61K 31/192; A61K 9/107; A61K 9/127

(57) ABSTRACT

Disclosed are methods of treating anti-inflammatory disease or conditions, typically by orally administering encochleated anti-inflammatory agents, including NSAIDS. Orally administered cochleates have significantly reduced toxicity as compared to non-encochleated anti-inflammatory agents.

### CROSS REFERENCE TO RELATED APPLICATIONS

This application claims the benefit of, and relies on the filing date of, U.S. Provisional Application No. 62/181,347, filed 18 Jun. 2015; U.S. Provisional Application No. 62/289,025, filed 29 Jan. 2016; and U.S. Provisional Application No. 62/347,014, filed 7 Jun. 2016, the entire disclosures of which are incorporated herein by reference.

### FIELD

This application relates generally to cochleate compositions and methods of administering the same to treat inflammatory disease or conditions.

### BACKGROUND

Anti-inflammatory drugs, such as non-steroidal anti-inflammatory drugs (NSAIDs) are some of the most commonly prescribed drugs in the world for their analgesic and anti-inflammatory properties. Unfortunately, NSAID use is limited by gastrointestinal (GI) toxicity. NSAIDs injure the gut by causing topical injury to the mucosa, including the initial development of lesions in the esophageal, gastric and intestinal sections of the GI tract, potentially culminating into peptic or intestinal ulcer disease and its complications, most notably upper gastrointestinal hemorrhage, and perforation, and by systemic effects associated with mucosal prostaglandin depletion derived from COX inhibition. As many as 25% of chronic NSAID users will develop ulcer disease and 2-4% will bleed or perforate. Lanza et al., Am. J Gastroenterol, 104:728-38 (2009). These gastrointestinal events result in more than 100,000 hospital admissions annually in the United States and between 7,000 and 10,000 deaths, especially among those who have been designated as being in a high-risk category. Lanza et al., Am. J Gastroenterol, 104:728-38 (2009). New formulations that prevent exposure of the NSAID to the GI mucosa may help reduce the GI toxicity associated with NSAIDs while maintaining their anti-inflammatory effects.

### SUMMARY

This application demonstrates that encochleated anti-inflammatory agents (e.g., NSAIDs) are more effective in vitro and in vivo than commercially available versions of the anti-inflammatory agents (e.g., NSAIDs). Further, the application demonstrates that encochleated anti-inflammatory agents (e.g., NSAIDs) are substantially safer and induce fewer gastric lesions or ulcers in the gastrointestinal tract. In addition, it was unexpectedly discovered that animals treated with geode cochleates, even at the highest dose tested, had no evidence of gastric lesions. By comparison, 9 of 10 animals treated with the highest dose of commercial NSAID and 6 of 10 treated with the highest dose of the NSAID-containing crystal cochleates contained gastric lesions, although animals treated with the NSAID-containing crystal cochleates still contained substantially fewer lesions and smaller lesions than those observed in animals treated with equal doses of the commercial NSAID.

Thus, the disclosure is directed, in part, to methods of treating a subject in need thereof with an encochleated anti-inflammatory agent (e.g., NSAID), wherein the subject has poor tolerability to a non-encochleated version of the anti-inflammatory agents (e.g., NSAID) or is susceptible to anti-inflammatory agent-induced (e.g., NSAID-induced) lesions or ulcers in the gastrointestinal tract, the method comprising orally administering to the subject a formulation comprising a cochleate, wherein the cochleate comprises a therapeutically effective amount of an anti-inflammatory agent (e.g., NSAID). In certain embodiments, the cochleate is a geode cochleate comprising: 1) a lipid monolayer comprising a negatively charged phospholipid, wherein the lipid monolayer surrounds a hydrophobic domain and the NSAID is dispersed within the hydrophobic domain; and 2) a lipid strata comprising alternating divalent cations and phospholipid bilayers comprising the negatively charged phospholipid, wherein the lipid monolayer is sequestered within the lipid strata. In certain embodiments, the hydrophobic domain is an oil, such as castor oil.

Another aspect is directed to methods of treating a subject in need thereof with an encochleated anti-inflammatory agent (e.g., NSAID), the method comprising orally administering to the subject a formulation comprising a geode cochleate comprising a therapeutically effective amount of an anti-inflammatory agent (e.g., NSAID). Typically, the geode cochleate comprises: 1) a lipid monolayer comprising a negatively charged phospholipid, wherein the lipid monolayer surrounds a droplet of castor oil and the anti-inflammatory agent (e.g., NSAID) is comprised within the droplet of castor oil; and 2) a lipid strata comprising alternating divalent cations and phospholipid bilayers comprising the negatively charged phospholipid, wherein the lipid strata is disposed about the lipid monolayer.

In certain embodiments, the NSAID is selected from the group consisting of a salicylate (such as aspirin [acetylsalicylic acid], diflunisal, salsalate or salicylic acid and other salicylates), a propionic acid derivative (such as ibuprofen, dexibuprofen, naproxen, fenoprofen, ketoprofen, dexketoprofen, flurbiprofen, oxaprozin, or loxoprofen), an acetic acid derivative (such as indomethacin, tolmetin, sulindac, etodolac, ketorolac, diclofenac, aceclofenac, or nabumetone), an enolic acid (-oxicam) derivative (such as piroxicam, meloxicam, tenoxicam, droxicam, lornoxicam, isoxicam, or phenylbutazone), an anthranilic acid derivative (e.g., a fenamate, such as mefenamic acid, meclofenamic acid, flufenamic acid, or tolfenamic acid), a selective COX-2 inhibitor (such as celecoxib, rofecoxib, valdecoxib, parecoxib, lumiracoxib, etoricoxib, or firocoxib), a sulfonanilide (such as nimesulide) or others (such as clonixin, licofelone or H-harpagide).

In certain embodiments, the NSAID is a propionic acid derivative, such as ibuprofen or naproxen. In certain embodiments, the NSAID is an acetic acid derivative, such as diclofenac. In certain embodiments, the NSAID is an enolic acid derivative, such as piroxicam or meloxicam.

In certain embodiments, the cochleate comprising the therapeutically effective amount of the NSAID does not induce any lesions in the gastrointestinal tract when administered to the subject.

In certain embodiments, the geode cochleate reduces the proportion of subjects developing lesions or ulcers in the gastrointestinal tract of a subject by over 40%, 50%, 60%, 70%, 80%, 90%, or 100% as compared to the proportion of subjects developing lesions or ulcers in the gastrointestinal tract when treated with a nonencochleated version of the NSAID.

In certain embodiments, the geode cochleate reduces the average number of lesions or ulcers in the gastrointestinal tract of a subject by more than 40, 50, 60, 70%, 80%, 90%, or 100% as compared to a nonencochleated version of the NSAID.

In certain embodiments, the geode cochleate reduces the average size of lesions or ulcers in the gastrointestinal tract of a subject by more than 50, 60 70%, 80%, 90%, or 100% as compared to a nonencochleated version of the NSAID.

In certain embodiments, the efficacious or indicated NSAID dose in the geode cochleate is reduced by more than 20%, 30%, 40%, 50%, 60%, 70%, 80%, or 90% as compared to the dose of a nonencochleated version of the same NSAID.

In certain embodiments, the subject has poor tolerability to a non-encochleated NSAID.

In certain embodiments, the subject is susceptible to NSAID-induced lesions or ulcers in the gastrointestinal tract.

In certain embodiments, the ratio between the hydrophobic domain (HD) and the phospholipid component of the geode cochleate (PPLGD) HD:PPLGD or the castor oil domain (COD) and phospholipid component of the geode cochleate (PPLGD) COD:PPLGD is 1:20 or less, 1:15 or less, 1:10 or less, 1:8 or less, 1:6 or less, 1:5 or less, 1:4 or less, 1:3.5 or less, 1:3 or less, 1:2.75 or less, 1:2.5 or less, 1:2.25 or less, 1:2 or less, 1:1.75 or less, 1:1.5 or less, 1:1.25 or less 1:1 or less.

In certain embodiments, the subject is administered no more than 3 g, 2.5 g, 2 g 1.5 g, 1.25 g, 1 g, 750 mg, 500 mg, 400 mg, 300 mg, 250 mg, 200 mg, 150 mg, or 100 mg per day of the cochleate.

In certain embodiments, the cochleate is administered once a day. In other embodiments, the cochleate is administered twice per day.

In certain embodiments, the method further comprises before the administering step, a step of identifying the subject as being susceptible to NSAID-induced lesions or ulcers in the gastrointestinal tract.

In certain embodiments, the formulation further comprises bile salts. In certain embodiments, the cochleate formulation contains 0.1 mM to 0.5 mM bile salts.

In certain embodiments, the subject is a mammal. In certain embodiments, the subject is a human.

In certain embodiments, the cochleate comprises one or more negatively charged lipids, wherein the one or more negatively charged lipids comprise between 40% to 70% of the total lipid in the cochleate. In certain embodiments, the one or more negatively charged lipids comprise between 40% to 70% of the total lipid in the non-hydrophobic domain component of the cochleate. In certain embodiments, the one or more negatively charged lipids are negatively charged phospholipids and comprise between 40% to 70% of the total phospholipid material in the cochleate or in the non-hydrophobic domain component of the cochleate. In certain embodiments, the one or more negatively charged lipids comprise between 50% to 60% of the total lipid in the cochleate. In certain embodiments, the one or more negatively charged lipids comprise between 50% to 60% of the total lipid in the non-hydrophobic domain component of the cochleate. In certain embodiments, the one or more negatively charged lipids are negatively charged phospholipids and comprise between 50% to 60% of the total phospholipid material in the cochleate.

In certain embodiments, the one or more negatively charged lipids comprise phosphatidylserine. In certain embodiments, the phosphatidylserine is soy phosphatidylserine.

In certain embodiments, the cochleate further comprise one or more neutral or cationic lipid or sterols. In certain embodiments, the one or more neutral or cationic lipid or sterols are selected from the group consisting of phosphatidylcholine and sphingomyelin.

### BRIEF DESCRIPTION OF THE DRAWINGS

The accompanying drawings, which are incorporated in and constitute a part of this specification, illustrate certain embodiments, and together with the written description, serve to explain certain principles of the compositions and methods disclosed herein.

FIG. 1 shows the in vitro efficacy of NSAID cochleates, as measured by nitrite concentration.

FIG. 2 shows that crystal and geode cochleates enhance the in vivo efficacy of ibuprofen in a carrageenan paw edema assay, as compared to a commercial preparation of ibuprofen, particularly at lower doses.

FIG. 3 shows an exemplary schematic of how geode cochleates can be made. In this exemplary method, a phospholipid (represented as an open ring) is combined with a hydrophobic domain (shaded circles), such as an oil, and mixed to form a stable emulsion comprising liposomes and lipid monolayers surrounding the hydrophobic domain. A cargo moiety, such as a drug, vitamin, NSAID, etc., may be dispersed within the hydrophobic domain. The hydrophobic domains have phospholipids imbedded in their surface. Without intending to be bound by any theory, it is believed that the hydrophobic acyl chains of the phospholipid are within the hydrophobic domains, resulting in the hydrophobic domains having a hydrophilic surface due to the coating of the phospholipid head groups and forming a stable emulsion. If the phospholipid is negatively charged, such as phosphatidylserine, the addition of a divalent cation, such as calcium, induces the formation of a crystalline structure (or lipid strata) comprising alternating divalent cations and phospholipid bilayers. The lipid strata are represented with hatching. In a geode cochleate, the lipid monolayers surrounding the hydrophobic domain are “encrusted” or “entrapped” within the crystalline matrix, akin to a “geode.”

### DETAILED DESCRIPTION

Reference will now be made in detail to various exemplary embodiments, examples of which are illustrated in the accompanying drawings and discussed in the detailed description that follows. It is to be understood that the following detailed description is provided to give the reader a fuller understanding of certain embodiments, features, and details of aspects of the invention, and should not be interpreted as limiting the scope of the invention.

### 1. Cochleates and Methods of Making the Same

Cochleates are anhydrous, stable, multi-layered lipid crystals which spontaneously form upon the interaction of negatively charged lipids, such as phosphatidylserine, and calcium (see, for example, U.S. Pat. Nos. 4,078,052; 5,643,574; 5,840,707; 5,994,318; 6,153,217; 6,592,894, as well as PCT Publ. Nos. WO 2004/091572; WO 2004/091578; WO 2005/110361, WO 2012/151517, and WO2014/022414, and U.S. Pat. Publ. 2010/0178325; each of which is incorporated fully herein by this reference). Typically, these are referred to as crystal cochleates. A variation of the crystal cochleate is known as the geode cochleate, or a geodate, as described, for example, in U.S. Pat. Publ. 2013/0224284, the entire disclosure of which is incorporated herein by reference.

Crystal and geode cochleates have a unique multilayered structure consisting of a large, continuous, solid, phospholipid bilayer sheet or strata rolled up in a spiral or as stacked sheets, with no internal aqueous space. This unique structure provides protection from degradation for associated “encochleated” molecules. Since the entire cochleate structure is a series of solid layers, components within the interior of the cochleate structure remain intact, even though the outer layers of the cochleate may be exposed to harsh environmental conditions or enzymes. Divalent cation concentrations in vivo in serum and mucosal secretions are such that the cochleate structure is maintained. Hence, the majority of cochleate-associated molecules are present in the inner layers of a solid, stable, impermeable structure. Once within the interior of a cell, however, the low calcium concentration results in the opening of the cochleate crystal and release of the molecule that had been formulated into cochleates. Accordingly, cochleate formulations remain intact in physiological fluids, including mucosal secretions, plasma and gastrointestinal fluid, thereby mediating the delivery of biologically active compounds by many routes of administration, including oral, mucosal and intravenous.

Typical cochleate structures include a lipid strata comprising alternating divalent cations and phospholipid bilayers that include at least one negatively charged phospholipid. Typically, a cargo moiety, such as a drug, vitamin, etc., is sequestered within the lipid strata of the cochleate. Geode cochleates further comprise a lipid monolayer comprising a negatively charged phospholipid, where the lipid monolayer surrounds a hydrophobic domain, such as an oil, and a cargo moiety, such as a drug, vitamin, etc., is dispersed within the hydrophobic domain. The lipid monolayer is sequestered within the lipid strata of the geode cochleate.

Cochleates can be made using known methods including, but not limited to, those described in U.S. Pat. Nos. 5,994,318 and 6,153,217, and U.S. Pat. Publ. 2013/0224284, the entire disclosures of which are incorporated herein by reference. In one embodiment, the method generally includes combining a pharmacologically active agent (e.g., an anti-inflammatory agent, such as an NSAID) with a lipid (preferably a negatively charged phospholipid, such as phosphatidylserine) in the presence of a solvent, adding an aqueous solution to form liposomes, and precipitating with a multivalent cation to form a cochleate. In another embodiment, the method generally includes combining a pharmacologically active agent (e.g., an anti-inflammatory agent, such as an NSAID) with a liposome in the presence of a solvent such that the pharmacologically active agent (e.g., an anti-inflammatory agent, such as an NSAID) associates with the liposome, and precipitating with a multivalent cation to form a pharmacologically active agent-containing cochleate.

In a preferred embodiment, the multivalent cation is a divalent metal cation, such as calcium, zinc, magnesium, and barium. In a preferred embodiment, the divalent metal cation is calcium.

The step of introducing a pharmacologically active agent (e.g., an anti-inflammatory agent, such as an NSAID) to a liposome in the presence of a solvent can be achieved in a variety of ways. In one embodiment, the pharmacologically active agent (e.g., an anti-inflammatory agent, such as an NSAID) is introduced by introducing a solution of the solvent and the pharmacologically active agent (e.g., an anti-inflammatory agent, such as an NSAID) to the liposome. Preferably, the liposome is in a liposomal suspension, preferably, an aqueous liposomal suspension. In a preferred embodiment, the solution with the anti-inflammatory agent is introduced to the liposome by dropwise addition of the solution. In other embodiments, the solution can be added by continuous flow or as a bolus. In addition the solution may be introduced to dried lipid, with water added before, after or with the solution.

In another embodiment, the pharmacologically active agent (e.g., an anti-inflammatory agent, such as an NSAID) is introduced to the liposome prior to or after the solvent. For example, the pharmacologically active agent (e.g., an anti-inflammatory agent, such as an NSAID) may be introduced to a liposomal suspension that includes the solvent. The mixture can then be agitated, mixed, vortexed or the like to facilitate association of the pharmacologically active agent (e.g., an anti-inflammatory agent, such as an NSAID) with the liposome. The pharmacologically active agent (e.g., an anti-inflammatory agent, such as an NSAID) introduced may be in a powder or a liquid form.

An antioxidant (e.g., Vitamin E) can also be used in making cochleates. The antioxidant can be introduced with the pharmacologically active agent (e.g., an anti-inflammatory agent, such as an NSAID) or with the liposome. Preferably, it is incorporated into the liposomal suspension or a solution of the pharmacologically active agent (e.g. an anti-inflammatory agent, such as an NSAID) and solvent.

The liposome may be prepared by any known method of preparing liposomes. Thus, the liposomes may be prepared for example by solvent injection, lipid hydration, reverse evaporation, freeze drying by repeated freezing and thawing. The liposomes may be multilamellar (MLV) or unilamellar (ULV), including small unilamellar vesicles (SUV). The concentration of lipid in these liposomal solutions can be from about 0.1 mg/ml to 500 mg/ml. Preferably, the concentration of lipid is from about 0.5 mg/ml to about 50 mg/ml, more preferably from about 1 mg/ml to about 25 mg/ml.

The liposomes may be large unilamellar vesicles (LUV), stable plurilamellar vesicles (SPLV) or oligolamellar vesicles (OLV) prepared, e.g., by detergent removal using dialysis, column chromatography, bio beads SM-2, by reverse phase evaporation (REV), or by formation of intermediate size unilamellar vesicles by high pressure extrusion. Methods in Biochemical Analysis, 33:337 (1988).

Any suitable solvent can be used in these methods. Solvents suitable for a given application can be readily identified by a person of skill in the art. Preferably, the solvent is an FDA acceptable solvent. The solvent can be an organic solvent or an inorganic solvent. In one embodiment, the solvent is a water miscible solvent. In another embodiment, the solvent is water or an aqueous buffer. Other suitable solvents include but are not limited to dimethylsulfoxide (DMSO), a methylpyrrolidone, N-methylpyrrolidone (NMP), acetonitrile, alcohols, e.g., ethanol (EtOH), dimethylformamide (DMF), tetrahydrofuran (THF), and combinations thereof. In general, the pharmacologically active agent (e.g., an anti-inflammatory agent, such as an NSAID) concentration within the solvent is between about 0.01 mg/ml and 200 mg/ml. Preferably, the pharmacologically active agent (e.g., an anti-inflammatory agent, such as an NSAID) concentration is between about 0.05 mg/ml and about 100 mg/ml, more preferably between about 0.1 mg/ml and 20 mg/ml.

The solvent can optionally be removed, e.g., before the formation of liposomes, at the liposome stage and/or after the cochleates are formed. Any known solvent removal method can be employed. For example, solvent may be removed from the liposomal suspension by tangential flow and/or filtration and/or dialysis, or from the cochleates by washing, filtration, centrifugation, and/or dialysis. The cochleates can be washed, e.g., with buffer or water, optimally with calcium or another cation.

A size-regulating agent may be introduced during the method of making the cochleate. A size-regulating agent, as used herein, refers to an agent that reduces the particle size of a cochleate. As used herein, the term “particle size” refers to the particle diameter, or in case the particles are not spherical, to the largest extension in one direction of the particle. The particle size of cochleates can be measured using conventional methods, such as a submicron particle size analyzer. In certain embodiments, the size regulating agent is a lipid-anchored polynucleotide, a lipid-anchored sugar (glycolipid), or a lipid-anchored polypeptide. In other embodiments the size regulating agent is a bile salt, such as oxycholate, cholate, chenodeoxycholate, taurocholate, glycocholate, taurochenodeoxycholate, glycochenodeoxycholate, deoxycholate, or lithocholate. Bile salts are bile acids compounded with a cation, usually sodium. Bile acids are steroid acids found predominantly in the bile of mammals and are commercially available.

In certain embodiments, the size-regulating agent is added to the lipid or liposomes before formation of the precipitated cochleate. For example, in one embodiment, the size-regulating agent is introduced into a liposomal suspension from which cochleates will subsequently be formed (e.g., by addition of cation or dialysis). Alternatively, the size-regulating agent may be introduced to a lipid solution, before or after addition of a pharmacologically active agent.

Any suitable lipid can be used to make the cochleate. In one embodiment, the lipid includes one or more negatively charged lipids. As used herein, the term “negatively charged lipid” includes lipids having a head group bearing a formal negative charge in aqueous solution at an acidic, basic or physiological pH, and also includes lipids having a zwitterionic head group.

The cochleates can also include non-negatively charged lipids (e.g., positive and/or neutral lipids). Preferably, the cochleates include a significant amount of negatively charged lipids. In certain embodiments, a majority of the lipid is negatively charged. In one embodiment, the lipid is a mixture of lipids, comprising at least 50% negatively charged lipid, such as a phospholipid. In another embodiment, the lipid includes at least 75% negatively charged lipid, such as a phospholipid. In other embodiments, the lipid includes at least 85%, 90%, 95% or 98% negatively charged lipid, such as a phospholipid. In yet other embodiments, the negatively charged lipid (e.g., phospholipid) comprises between 30%-70%, 35%-70%, 40%-70%, 45%-65%, 45%-70%, 40%-60%, 50%-60%, 45%-55%, 45%-65%, or 45%-50% of the total lipid in the cochleate. In certain embodiments, the negatively charged lipid (e.g., phospholipid) comprises between 40%-60% or 45%-55% of the total lipid in the cochleate. In some embodiments, the negatively charged lipid (e.g., phospholipid) comprises between 30%-70%, 35%-70%, 40%-70%, 45%-65%, 45%-70%, 40%-60%, 50%-60%, 45%-55%, 45%-65%, or 45%-50% of the total lipid in the non-hydrophobic domain component of the cochleate. In certain embodiments, the negatively charged lipid (e.g., phospholipid) comprises between 40%-60% or 45%-55% of the total lipid in the non-hydrophobic domain component of the cochleate. In some embodiments, the negatively charged lipid is a phospholipid and comprises between 30%-70%, 35%-70%, 40%-70%, 45%-65%, 45%-70%, 40%-60%, 50%-60%, 45%-55%, 45%-65%, or 45%-50% of the total phospholipid in the cochleate or in the non-hydrophobic domain component of the cochleate. In some embodiments, the negatively charged lipid is a phospholipid and comprises between 40%-60% or 45%-55% of the total phospholipid in the cochleate or in the non-hydrophobic domain component of the cochleate.

The negatively charged lipid can include soy-based lipids, other-legume-based lipids, egg-based lipids, bovine-based lipids, porcine-based lipids, or similar lipids derived from other sources. Preferably, the lipid includes phospholipids, such as soy-based phospholipids. The negatively charged lipid can include phosphatidylserine (PS), dioleoylphosphatidylserine (DOPS), phosphatidic acid (PA), phosphatidylinositol (PI), and/or phosphatidyl glycerol (PG) and or a mixture of one or more of these lipids with other lipids. Additionally or alternatively, the lipid can include phosphatidylcholine (PC), phosphatidylethanolamine (PE), diphosphotidylglycerol (DPG), dioleoyl phosphatidic acid (DOPA), di stearoyl phosphatidylserine (DSPS), dimyristoyl phosphatidylserine (DMPS), dipalmitoyl phosphatidylglycerol (DPPG) and the like. In one embodiment, the phosphatidylserine is soy phosphatidylserine. In another embodiment, the phosphatidylserine is egg or bovine derived phosphatidylserine.

As used herein, a “hydrophobic domain” is a composition that is sufficiently hydrophobic in nature to allow formation of a lipid monolayer about its periphery. A hydrophobic domain typically includes a hydrophobic composition, such as oil or fat, associated with a cargo moiety, such as an anti-inflammatory agent (e.g., NSAID). In certain embodiments, the ratio between the hydrophobic domain (HD) and the phospholipid component of the geode cochleate (PPLGD) HD:PPLGD or the castor oil domain (COD) and phospholipid component of the geode cochleate (PPLGD) COD:PPLGD is 1:20 or less, 1:15 or less, 1:10 or less, 1:8 or less, 1:6 or less, 1:5 or less, 1:4 or less, 1:3.5 or less, 1:3 or less, 1:2.75 or less, 1:2.5 or less, 1:2.25 or less, 1:2 or less, 1:1.75 or less, 1:1.5 or less, 1:1.25 or less 1:1 or less.

### 2. Anti-Inflammatory Agents

The cochleates for use in the methods described herein are associated with or loaded with an anti-inflammatory agent. By way of example, the anti-inflammatory agent can include, but is not limited to, one or more of the following: an NSAID, including an NSAID which belongs to is one or more of the following classes: a Salicylate (such as aspirin [acetylsalicylic acid], diflunisal, salsalate or salicylic acid and other salicylates), a propionic acid derivative (such as ibuprofen, dexibuprofen, naproxen, fenoprofen, ketoprofen, dexketoprofen, flurbiprofen, oxaprozin, or loxoprofen), an acetic acid derivative (such as indomethacin, tolmetin, sulindac, etodolac, ketorolac, diclofenac, aceclofenac, or nabumetone), an enolic acid (-oxicam) derivative (such as piroxicam, meloxicam, tenoxicam, droxicam, lornoxicam, isoxicam, or phenylbutazone), an anthranilic acid derivative (e.g., a fenamate, such as mefenamic acid, meclofenamic acid, flufenamic acid, or tolfenamic acid), a selective COX-2 inhibitor (such as celecoxib, rofecoxib, valdecoxib, parecoxib, lumiracoxib, etoricoxib, or firocoxib), a sulfonanilide (such as nimesulide) or others (such as clonixin, licofelone or H-harpagide).

In other embodiments, the anti-inflammatory agent can include, but is not limited to, one or more of the following: a corticosteroid (such as hydrocortisone, hydrocortisone acetate, cortisone acetate, tixocortol pivalate, prednisolone, methylprednisolone, prednisone, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-valerate, halometasone, alclometasone dipropionate, betamethasone valerate, betamethasone dipropionate, prednicarbate, clobetasone-17-butyrate, clobetasol-17-propionate, fluocortolone caproate, fluocortolone pivalate, fluprednidene acetate, hydrocortisone-17-butyrate, hydrocortisone-17-aceponate, hydrocortisone-17-buteprate, ciclesonide, prednicarbate, flunisolide, fluticasone furoate, fluticasone propionate, triamcinolone acetonide, beclomethasone dipropionate, and budesonide), a DMARD (such as methotrexate, azathioprine, ciclosporin, penicillamine, auranofin, aurothiomalate salts, minocycline, hydroxychloroquine, chloroquine, sulfasalazine, leflunomide, teriflunomide, mesalamine, or cyclophosphamide), acetaminophen, an anti-TNF agent (such as adalimumab, infliximab, etanercept, certolizumab pegol), a macrolide calcineruin inhibitor (such as sirolimus or tacrolimus), a JAK-inhibitor (such as tofacitinib, Ruxolitinib, Baricitinib (LY3009104, INCB28050), CYT387, Filgotinib (GLPG0634), GSK2586184, Lestaurtinib, Pacritinib (SB1518), TG101348, JSI-124, or CHZ868), an IL-6 antagoinst (such as tocilizumab or atlizumab), an anti-CD20 agent (such as rituximab, obinutuzumab, Ibritumomab tiuxetan, tositumomab, ofatumumab, ocrelizumab, TRU-015, or IMMU-106 [veltuzumab]), a CD52-antagonist (such as alemtuzumab), an alpha-4 integrin antagonist (such as natalizumab), a type II topoisomerase inhibitor (such as mitoxantrone), a sphingosine-1-phosphate receptor modulator (such as fingolimod, laquinimod, ozanimod, or ponesimod), a beta-interferon, or one of the following agents or their functional similars: glatiramer acetate or dimethyl fumarate.

In certain embodiments, the anti-inflammatory agent is an NSAID. In certain embodiments, the NSAID is a propionic acid derivative. In certain embodiments, the propionic acid derivative is ibuprofen or naproxen. In certain embodiments, the propionic acid derivative is naproxen. In certain embodiments, the anti-inflammatory agent is an acetic acid derivative. In certain embodiments, the acetic acid derivative is diclofenac. In certain embodiments, the anti-inflammatory agent is an enolic acid derivative. In certain embodiments, the enolic acid derivative is piroxicam or meloxicam. In certain embodiments, the NSAID is a selective COX-2 inhibitor. In certain embodiments the selective COX-2 inhibitor is celecoxib or rofecoxib.

### 3. Pharmaceutical Compositions

The cochleates described herein can be prepared as a pharmaceutical composition. Suitable preparation forms for the pharmaceutical compositions disclosed herein include, for example, tablets, capsules, soft capsules, granules, powders, suspensions, emulsions, microemulsions, nanoemulsions, unit dosage forms, rings, films, suppositories, solutions, creams, syrups, transdermal patches, ointments and gels.

The pharmaceutical compositions can include other pharmaceutically acceptable excipients, such as, a buffer (e.g., Tris-HCl, acetate, phosphate) of various pH and ionic strength; an additive such as albumin or gelatin to prevent absorption to surfaces; a protease inhibitor; a permeation enhancer; a solubilizing agent (e.g., glycerol, polyethylene glycerol); an anti-oxidant (e.g., ascorbic acid, sodium metabisulfite, butylated hydroxyanisole); a stabilizer (e.g., hydroxypropyl cellulose, hydroxypropylmethyl cellulose); a viscosity increasing agent (e.g., carbomer, colloidal silicon dioxide, ethyl cellulose, guar gum); a sweetener (e.g. aspartame, citric acid); a preservative (e.g., Thimerosal, benzyl alcohol, parabens); a flow-aid (e.g., colloidal silicon dioxide), a plasticizer (e.g., diethyl phthalate, triethyl citrate); an emulsifier (e.g., carbomer, hydroxypropyl cellulose, sodium lauryl sulfate); a polymer coating (e.g., poloxamers or poloxamines, hypromellose acetate succinate); a coating and film forming agent (e.g., ethyl cellulose, acrylates, polymethacrylates, hypromellose acetate succinate); an adjuvant; a pharmaceutically acceptable carrier for liquid formulations, such as an aqueous (water, alcoholic/aqueous solution, emulsion or suspension, including saline and buffered media) or non-aqueous (e.g., propylene glycol, polyethylene glycol, and injectable organic esters such as ethyl oleate) solution, suspension, emulsion or oil; and a parenteral vehicle (for subcutaneous, intravenous, intraarterial, or intramuscular injection), including but not limited to, sodium chloride solution, Ringer's dextrose, dextrose and sodium chloride, lactated Ringer's and fixed oils.

In certain embodiments, the pharmaceutical composition comprises a salt, such as NaCl or a bile salt, such as oxycholate, cholate, chenodeoxycholate, taurocholate, glycocholate, taurochenodeoxycholate, glycochenodeoxycholate, deoxycholate, or lithocholate. Bile salts are bile acids compounded with a cation, usually sodium. Bile acids are steroid acids found predominantly in the bile of mammals and are commercially available. In one embodiment, the bile salts comprise cholate. In another embodiment, the bile salts comprises deoxycholate. In yet another embodiment, the bile salts comprise cholate and deoxycholate. In another embodiment, the bile salts consist of cholate and deoxycholate.

In certain embodiments, the concentration of NaCl is 1 mM to 1M, 1 mM to 0.5M, 1 mM to 0.1M, 1 mM to 50 mM, 10 mM to 100 mM, 10 mM to 50 mM, 0.1M to 1M, 0.1M to 0.5M, or 0.5M to 1M. In certain embodiments, the concentration of the bile salts is 1 mM to 100 mM, 1 mM to 50 mM, 1 mM to 25 mM, 1 mM to 10 mM, 1 mM to 5 mM, 0.1 mM to 5 mM, 0.1 mM to 1 mM, or 0.1 mM to 0.5 mM bile salts.

These excipients are provided by way of example and it will be known to those of skill in the art that there will be other or different excipients that can provide the same chemical features as those listed herein.

### 4. Dosage and Administration

A pharmaceutical composition comprising a cochleate, as disclosed herein, is formulated to be compatible with its intended route of administration. Methods to accomplish the administration are known to those of ordinary skill in the art. This includes, for example, injections, by parenteral routes such as intravenous, intravascular, intraarterial, subcutaneous, intramuscular, intraperitoneal, intraventricular, intraepidural, or others as well as oral, nasal, ophthalmic, rectal, or topical. Typically, the cochleate is administered orally, for example, by administering a suspension, a tablet, a capsule, a softgel or other oral dosage form.

In certain embodiments, the anti-inflammatory (e.g., an NSAID) is administered at a dosage of between 0.05-1 mg/kg, 0.1-2 mg/kg, 0.2-3 mg/kg, 0.5-5 mg/kg, 1-5 mg/kg, 1-10 mg/kg, 2-5 mg/kg, 2-10 mg/kg, 3-15 mg/kg, 5-20 mg/kg, 5-10 mg/kg, 5-15 mg/kg, 10-15 mg/kg, 10-13 mg/kg, 10-20 mg/kg, 5-25 mg/kg, 1-30 mg/kg, 20-100 mg/kg, 30-60 mg/kg, or 30-50 mg/kg. Alternatively, the anti-inflammatory (e.g., an NSAID) can be administered at a fixed dosage of about 5-50 mg/day, 10-50 mg/day, 20-100 mg/day, 50-200 mg/day, 100-500 mg/day, 200-1000 mg/day, 400-1000 mg/day, 200-800 mg/day, 300-800 mg/day, 400-800 mg/day, 500-700 mg/day, 200-2000 mg/day, 100-6400 mg/day, 100-600 mg/day, 200-600 mg/day, 400-600 mg/day, 300-700 mg/day, 700-1200, 1000-3000, or 2000-4000, including, but not limited to about 400, 500, 600, 700, 800, 900, 1000, 1500, 2000, 2500, 3000, 3500, 4000, 4500, 5000, 6000, or 6400 mg.

Due to lower toxicity, the encochleated anti-inflammatory (e.g., NSAID) may be administered at a higher dosage, more frequently, or for a longer duration than a non-encochleated version of the anti-inflammatory (e.g., NSAID). In certain embodiments, the encochleated anti-inflammatory (e.g., NSAID) is administered at a higher dosage than a non-encochleated version of the anti-inflammatory (e.g., NSAID). In certain embodiments, the higher dosage is about 25-300% greater than the recommended or indicated dosage of a non-encochleated version of the anti-inflammatory (e.g., NSAID). In certain embodiments, higher dosage is about 50-200%, 25-100% or 25-50% greater than the recommended or indicated dosage of a non-encochleated version of the anti-inflammatory (e.g., NSAID). Alternatively, the anti-inflammatory (e.g., NSAID) dose in the cochleate is increased by more than 20%, 30%, 40%, 50%, 60% 70%, 80%, 90%, 100%, 150%, or 200% as compared to the recommended or indicated dose of a nonencochleated version of the same anti-inflammatory (e.g., NSAID). The recommended or indicated dosage of an anti-inflammatory is common knowledge in the art and may be adjusted depending on the route of administration and the physical characteristics of the patient, such as general state, age, weight, diet, and other medications. In certain embodiments, the encochleated anti-inflammatory (e.g., NSAID) may be administered once per day, twice per day, three times per day, or four times per day. In other embodiments, the cochleate formulation is administered daily for at least 1, 2, 3, 4, 5, 6, 7, 8, 9, 10, 11, or 12 weeks. In another embodiment, the cochleate formulation is administered daily for at least 3 months, at least 4 months, or at least 6 months.

Due to its enhanced pharmacokinetics, which may be due in part to enhanced tissue penetration, the encochleated anti-inflammatory (e.g., NSAID) may also be administered at a lower dosage, less frequently, or for a shorter duration than a non-encochleated version of the anti-inflammatory (e.g., NSAID). In certain embodiments, the lower dosage is about 25-300% less than the recommended or indicated dosage of a non-encochleated version of the anti-inflammatory (e.g., NSAID). In certain embodiments, lower dosage is about 50-200%, 25-100% or 25-50% less than the recommended or indicated dosage of a non-encochleated version of the anti-inflammatory (e.g., NSAID). Alternatively, the anti-inflammatory (e.g., NSAID) dose in the cochleate is reduced by more than 20%, 30%, 40%, 50%, 60% 70%, 80%, or 90% as compared to the recommended or indicated dose of a nonencochleated version of the same anti-inflammatory (e.g., NSAID). The recommended or indicated dosage of an anti-inflammatory is common knowledge in the art and may be adjusted depending on the route of administration and the physical characteristics of the patient, such as general state, age, weight, diet, and other medications. In another embodiment, the cochleate formulation is administered once per week, twice per week, three times per week, or four times per week. In one embodiment, the encochleated anti-inflammatory (e.g., NSAID) may be administered 2-3 times weekly.

Typically, the recommended or indicated dosage of non-encochleated ibuprofen for minor inflammation (e.g., minor pain or fever) is about 200 mg every 4-6 hours and a daily maximum dosage of 1200 mg. In one embodiment, the NSAID in the cochleate is ibuprofen and the encochleated ibuprofen is administered for minor inflammation at a dosage of about 25-300%, 50-200%, 25-100%, or 25-50% greater than the recommended or indicated dosage of non-encochleated ibuprofen. In another embodiment, the NSAID in the cochleate is ibuprofen and the encochleated ibuprofen is administered for minor inflammation at a dosage of about 25-300%, 50-200%, 25-100%, or 25-50% less than the recommended or indicated dosage of non-encochleated ibuprofen.

Typically, the recommended or indicated dosage of non-encochleated ibuprofen for other inflammatory conditions (e.g., dysmenorrhea, rheumatoid arthritis, osteoarthritis) is about 1200-3200 mg every day. In one embodiment, the NSAID in the cochleate is ibuprofen and the encochleated ibuprofen is administered for inflammatory conditions other than minor inflammation at a dosage of about 25-300%, 50-200%, 25-100%, or 25-50% less than the recommended or indicated dosage of non-encochleated ibuprofen. In another embodiment, the NSAID in the cochleate is ibuprofen and the encochleated ibuprofen is administered for inflammatory conditions other than minor inflammation at a dosage of about 25-300%, 50-200%, 25-100%, or 25-50% less than the recommended or indicated dosage of non-encochleated ibuprofen.

Typically, the recommended or indicated dosage of non-encochleated naproxen for minor inflammation (e.g., minor pain or fever) is about 220 mg every 8-12 hours and a daily maximum dosage of 660 mg. In one embodiment, the NSAID in the cochleate is naproxen and the encochleated naproxen is administered for minor inflammation at a dosage of about 25-300%, 50-200%, 25-100%, or 25-50% greater than the recommended or indicated dosage of non-encochleated naproxen. In another embodiment, the NSAID in the cochleate is naproxen and the encochleated naproxen is administered for minor inflammation at a dosage of about 25-300%, 50-200%, 25-100%, or 25-50% less than the recommended or indicated dosage of non-encochleated naproxen.

Typically, the recommended or indicated dosage of non-encochleated naproxen for other inflammatory conditions (e.g., dysmenorrhea, bursitis, tendonitis, rheumatoid arthritis, ankylosing spondylitis) is about 500-1000 mg every day and a daily maximum dosage of up to 1500 mg. In one embodiment, the NSAID in the cochleate is naproxen and the encochleated naproxen is administered for inflammatory conditions other than minor inflammation at a dosage of about 25-300%, 50-200%, 25-100%, or 25-50% greater than the recommended or indicated dosage of non-encochleated naproxen. In another embodiment, the NSAID in the cochleate is naproxen and the encochleated naproxen is administered for inflammatory conditions other than minor inflammation at a dosage of about 25-300%, 50-200%, 25-100%, or 25-50% less than the recommended or indicated dosage of non-encochleated naproxen.

Typically, the recommended or indicated dosage of non-encochleated diclofenac for minor inflammation (e.g., minor pain or fever) is about 25 mg every 6 hours and a daily maximum dosage of about 100 mg. In one embodiment, the NSAID in the cochleate is diclofenac and the encochleated diclofenac is administered for minor inflammation at a dosage of about 25-300%, 50-200%, 25-100%, or 25-50% greater than the recommended or indicated dosage of non-encochleated diclofenac. In another embodiment, the NSAID in the cochleate is diclofenac and the encochleated diclofenac is administered for minor inflammation at a dosage of about 25-300%, 50-200%, 25-100%, or 25-50% less than the recommended or indicated dosage of non-encochleated diclofenac.

Typically, the recommended or indicated dosage of non-encochleated diclofenac for other inflammatory conditions (e.g., dysmenorrhea, rheumatoid arthritis, osteoarthritis, ankylosing spondylitis) is about 35-75 mg 2-3 times per day and a daily maximum dosage of up to 225 mg. In one embodiment, the NSAID in the cochleate is diclofenac and the encochleated diclofenac is administered for inflammatory conditions other than minor inflammation at a dosage of about 300%, 50-200%, 25-100%, or 25-50% greater than the recommended or indicated dosage of non-encochleated diclofenac. In another embodiment, the NSAID in the cochleate is diclofenac and the encochleated diclofenac is administered for inflammatory conditions other than minor inflammation at a dosage of about 300%, 50-200%, 25-100%, or 25-50% less than the recommended or indicated dosage of non-encochleated diclofenac.

Typically, the recommended or indicated dosage of non-encochleated piroxicam for inflammatory conditions other than minor inflammation (e.g., osteoarthritis, rheumatoid arthritis) is about 20 mg/day. In one embodiment, the NSAID in the cochleate is piroxicam and the encochleated piroxicam is administered for inflammatory conditions other than minor inflammation at a dosage of about 25-300%, 50-200%, 25-100%, or 25-50% greater than the recommended or indicated dosage of non-encochleated piroxicam. In another embodiment, the NSAID in the cochleate is piroxicam and the encochleated piroxicam is administered for minor inflammation at a dosage of about 25-300%, 50-200%, 25-100%, or 25-50% less than the recommended or indicated dosage of non-encochleated piroxicam.

Typically, the recommended or indicated dosage of non-encochleated meloxicam for inflammatory conditions other than minor inflammation (e.g., osteoarthritis, rheumatoid arthritis) is an initial dose of 7.5 mg/day, followed by a maintenance dose of 15 mg/day, with a maximum daily dosage of 15 mg/day. In one embodiment, the NSAID in the cochleate is meloxicam and the encochleated meloxicam is administered for inflammatory conditions other than minor inflammation at a dosage of about 25-300%, 50-200%, 25-100%, or 25-50% greater than the recommended or indicated dosage of non-encochleated meloxicam. In another embodiment, the NSAID in the cochleate is meloxicam and the encochleated meloxicam is administered for minor inflammation at a dosage of about 25-300%, 50-200%, 25-100%, or 25-50% less than the recommended or indicated dosage of non-encochleated meloxicam.

Typically, the recommended or indicated dosage of non-encochleated celecoxib for minor inflammation (e.g., minor pain or dysmenorrhea) is an initial dose of about 400 mg followed by about 200 mg every 12 hours. In one embodiment, the NSAID in the cochleate is celecoxib and the encochleated celecoxib is administered for minor inflammation at a dosage of about 25-300%, 50-200%, 25-100%, or 25-50% greater than the recommended or indicated dosage of non-encochleated celecoxib. In another embodiment, the NSAID in the cochleate is celecoxib and the encochleated celecoxib is administered for minor inflammation at a dosage of about 25-300%, 50-200%, 25-100%, or 25-50% less than the recommended or indicated dosage of non-encochleated celecoxib.

Typically, the recommended or indicated dosage of non-encochleated celecoxib for inflammatory conditions other than minor inflammation (e.g., osteoarthritis, rheumatoid arthritis, ankylosing spondylitis) is about 200-400 mg/day. In one embodiment, the NSAID in the cochleate is celecoxib and the encochleated celecoxib is administered for inflammatory conditions other than minor inflammation at a dosage of about 25-300%, 50-200%, 25-100%, or 25-50% greater than the recommended or indicated dosage of non-encochleated celecoxib. In another embodiment, the NSAID in the cochleate is celecoxib and the encochleated celecoxib is administered for minor inflammation at a dosage of about 25-300%, 50-200%, 25-100%, or 25-50% less than the recommended or indicated dosage of non-encochleated celecoxib.

Typically, the recommended or indicated dosage of non-encochleated rofecoxib for minor inflammation (e.g., minor pain, migraine, or dysmenorrhea) is 25-50 mg/day for up to 5 days. In one embodiment, the NSAID in the cochleate is rofecoxib and the encochleated rofecoxib is administered for minor inflammation at a dosage of about 25-300%, 50-200%, 25-100%, or 25-50% greater than the recommended or indicated dosage of non-encochleated rofecoxib. In another embodiment, the NSAID in the cochleate is rofecoxib and the encochleated rofecoxib is administered for minor inflammation at a dosage of about 25-300%, 50-200%, 25-100%, or 25-50% less than the recommended or indicated dosage of non-encochleated rofecoxib.

Typically, the recommended or indicated dosage of non-encochleated rofecoxib for inflammatory conditions other than minor inflammation (e.g., osteoarthritis, rheumatoid arthritis) is about 12.5-25 mg/day (osteoarthritis) or 25-50 mg/day (rheumatoid arthritis). In one embodiment, the NSAID in the cochleate is rofecoxib and the encochleated rofecoxib is administered for inflammatory conditions other than minor inflammation at a dosage of about 25-300%, 50-200%, 25-100%, or 25-50% greater than the recommended or indicated dosage of non-encochleated rofecoxib. In another embodiment, the NSAID in the cochleate is rofecoxib and the encochleated rofecoxib is administered for minor inflammation at a dosage of about 25-300%, 50-200%, 25-100%, or 25-50% less than the recommended or indicated dosage of non-encochleated rofecoxib.

### 5. Methods of Treatment

The cochleates as described herein can be used in a method of treating a subject with an inflammatory disease or condition. Any inflammatory disease or condition can be treated including disease or condition for which treatment with an anti-inflammatory agent, such as an NSAID, is indicated. In certain embodiments, the inflammatory disease or condition includes, but is not limited to arthritis (e.g., osteoarthritis, rheumatoid arthritis, and juvenile rheumatoid arthritis), ankylosing spondylitis, familial adenomatous polyposis, primary dysmenorrhea, tendonitis, bursitis, gout, acute pain, fever, headache, toothache, or minor injury.

In certain embodiments, the method of treating a subject with an inflammatory disease or condition comprises orally administering to the subject in need thereof a therapeutically effective amount of a formulation comprising a cochleate, wherein the cochleate comprises an anti-inflammatory agent (e.g., NSAID).

One embodiment is directed to a method of treating a subject in need thereof with an encochleated anti-inflammatory agent, wherein the subject has poor tolerability to a non-encochleated anti-inflammatory agent or is susceptible to anti-inflammatory agent-induced lesions and/or ulcerations in the gastrointestinal tract, the method comprising orally administering to the subject a formulation comprising a cochleate, wherein the cochleate comprises a therapeutically effective amount of an anti-inflammatory agent. Typically, the anti-inflammatory agent is an NSAID as discussed elsewhere in this application. As used herein, “poor tolerability to a non-encochleated anti-inflammatory agent,” indicates that the subject has a history of NSAID-induced gastric lesions or ulcers. As used herein, “susceptible to anti-inflammatory agent-induced lesions in the gastrointestinal tract,” includes both “high risk” and “moderate risk” subjects as described below.

Another embodiment is directed to a method of treating a subject in need thereof with an encochleated anti-inflammatory agent, the method comprising orally administering to the subject a formulation comprising a cochleate, wherein the cochleate comprises a therapeutically effective amount of an anti-inflammatory agent, and wherein the cochleate is a geode cochleate comprising: 1) a lipid monolayer comprising a negatively charged phospholipid, wherein the lipid monolayer surrounds a droplet of castor oil and the anti-inflammatory agent is comprised within the droplet of castor oil; and 2) a lipid strata comprising alternating divalent cations and phospholipid bilayers comprising the negatively charged phospholipid, wherein the lipid strata is disposed about the lipid monolayer. Typically, the anti-inflammatory agent is an NSAID as discussed elsewhere in this application.

In certain embodiments, the treatment methods further comprise before the administering step, a step of identifying the subject as being susceptible to NSAID-induced lesions or ulcers in the gastrointestinal tract. Typically, the step comprises identifying in a subject one or more risk factors for NSAID-related GI complications. Risk factors for NSAID-related GI complications include, but are not limited to, a previous GI event, age, concomitant use of anticoagulants, corticosteroids, or other NSAIDs including low-dose aspirin, high-dose NSAID therapy, chronic debilitating disorders, such as cardiovascular disease, and H. pylori infection. Lanza et al., Am. J Gastroenterol, 104:728-38 (2009). Subjects can be further stratified into high, moderate or low risk as illustrated by way of example in the following stratification:

High Risk
1. History of a previously complicated ulcer, especially recent; or
2. Multiple risk factors (at least 2)

Moderate Risk (1-2 of the Following Risk Factors)
1. Older than 65 years;
2. High dose NSAID therapy;
3. A previous history of uncomplicated ulcer; and/or
4. Concurrent use of aspirin (including low dose) corticosteroids or anticoagulants

Low Risk
1. No risk factors

In certain embodiments, the subject is identified as being at high risk for developing NSAID-induced lesions or ulcers before beginning treatment with the encochleated NSAID. In certain embodiments, the subject is identified as being at moderate risk for developing NSAID-induced lesions or ulcers before beginning treatment with the encochleated NSAID. In certain embodiments, the subject has experienced NSAID-induced lesions or ulcers in the gastrointestinal tract due to treatment with a non-encochleated NSAID before beginning treatment with the encochleated NSAID.

In certain embodiments, the cochleate comprising the anti-inflammatory agent (e.g., NSAID) is administered as monotherapy. In other embodiments, the cochleate comprising the anti-inflammatory agent (e.g., NSAID) is administered as part of a multi-drug therapy, including for example at least one other anti-inflammatory or an agent for treating a NSAID-induced lesion or ulcer, such as misoprostol, a proton pump inhibitor, a high dose histamine-2-receptor antagonist, or a COX-2 inhibitor.

Typically, the subject is a human or a non-human mammal, such as a dog, a cat, a horse, or a farm animal. Typically, the subject is a human.

### 6. Reduced Toxicity

Oral administration of encochleated anti-inflammatory agents (e.g., NSAIDs), exhibits reduced toxicity as compared to oral administration of non-encochleated anti-inflammatory agents. As discussed above, oral administration of non-encochleated NSAIDs often results in NSAID-induced lesions, ulcers, and/or bleeding in the GI tract, especially for patients who are at moderate or high risk for developing NSAID-induced lesions, ulcers, and/or bleeding. The COX-2 inhibition mode of activity of NSAIDs can also lead to adverse cardiovascular events, including thrombolytic events.

In one embodiment, the encochleated NSAID reduces the proportion of subjects developing lesions or ulcers in the gastrointestinal tract by over 40% 50%, 60%, 70%, 80%, 90%, or by 100% as compared to the proportion of subjects developing lesions or ulcers in the gastrointestinal tract when treated with a nonencochleated version of the NSAID. Typically, the cochleate is a geode cochleate.

In one embodiment, the encochleated NSAID reduces the average number of lesions in the gastrointestinal tract of a subject by more than 40%, 50%, 60%, 70%, 80%, 90%, or by 100% as compared to a nonencochleated version of the NSAID. Typically, the cochleate is a geode cochleate.

In one embodiment, the encochleated NSAID reduces the average size of lesions in the gastrointestinal tract of a subject by more than 40%, 50%, 60%, 70%, 80%, 90%, or by 100% as compared to a nonencochleated version of the NSAID. Typically, the cochleate is a geode cochleate.

### EXAMPLES

The examples provided below are simply for illustrative purposes. Those of skill in the art will be able to readily determine appropriate methods and equipment in order to produce suitable solid dispersion forms as described herein.

### Example 1: In Vitro Efficacy of NSAID Cochleates

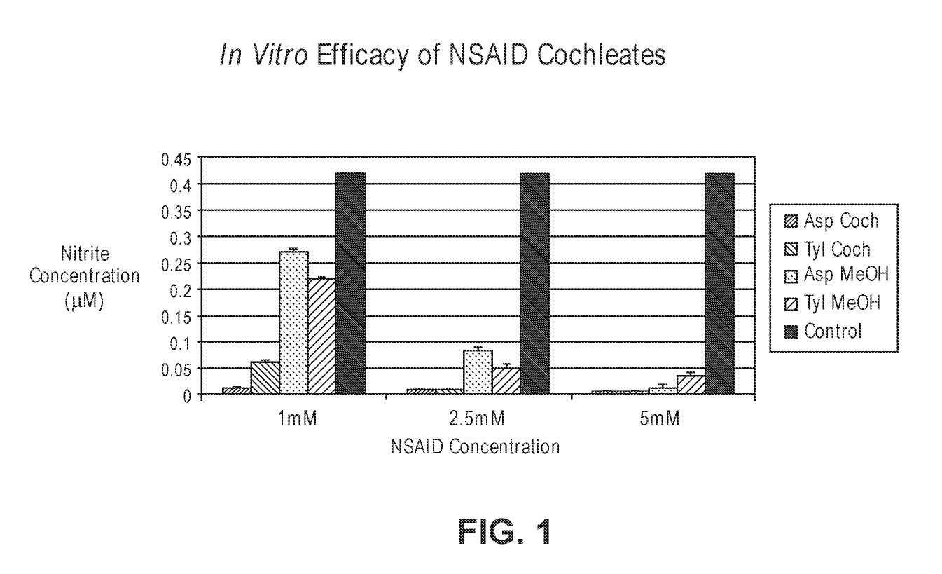

Crystal and geode cochleates were prepared containing 1 mM, 2.5 mM, and 5 mM of aspirin or TYLENOL and compared to a methanol preparation containing the same amounts of either aspirin or TYLENOL or a control. As shown in FIG. 1, the in vitro activity of encochleated NSAIDS was 5- and 10-fold greater than the activity of TYLENOL and aspirin, respectively, as measured by nitrite concentration.

### Example 2: In Vivo Efficacy of NSAID Cochleates

The in vivo efficacy if NSAID cochleates was tested in a rat carrageenan-induced paw inflammation assay. The carrageenan paw edema assay has long been used as an animal model to detect anti-inflammatory activity which suppresses prostaglandin production but other inflammatory mediators are important in the pathogenesis of the lesion. See Winter et al., Proc. Soc. Exp. Biol. Med.; 111:544-547, (1962); Van Armen C G et al. J Pharmacol Exp Ther. 150:328-334, (1965); Otterness I G et al., Laboratory models for testing nonsteroidal anti-inflammatory drugs. In nonsteroidal anti-inflammatory drugs, ed. By J G Lombardino, pp. 116-129, John Wiley & Sons, New York, (1985); and Vinegar R et al., Fed Proc 46:118-126, (1987). Generally the assay involves footpad injection of the irritant substance carrageenan, usually 0.5 to 1 hour after dosing with the test compound.

Methods:

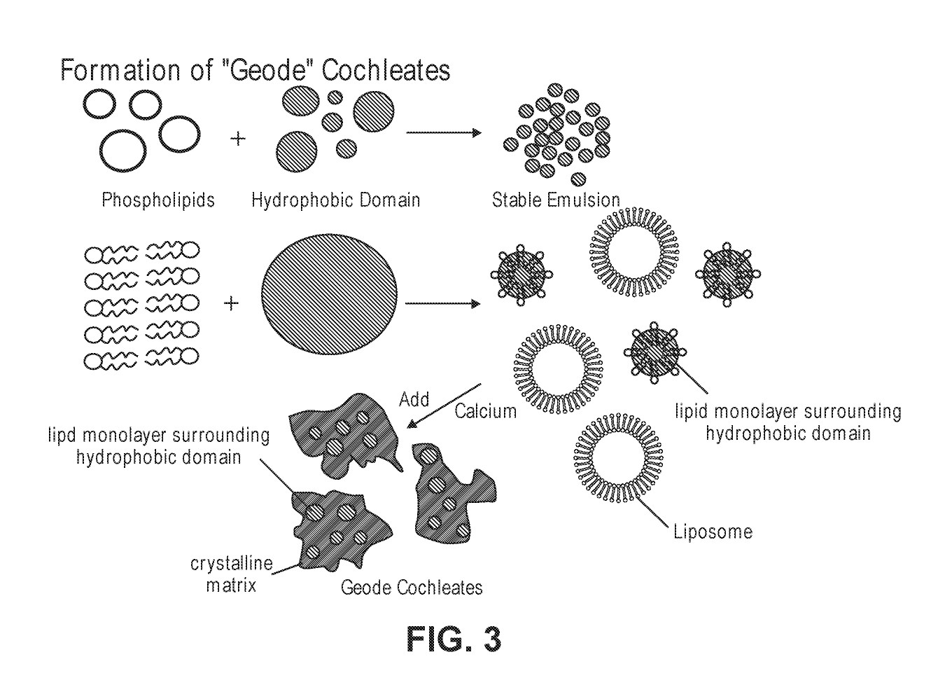

Two types of ibuprofen-containing cochleates were prepared, crystal cochleates and geode cochleates. The cochleates were prepared with using soy phosphatidylserine as a negative phospholipid. To prepare the ibuprofen geode cochleate, the soy phosphatidylserine was combined with castor oil and ibuprofen and mixed to form a stable emulsion comprising a mixture of liposomes and lipid monolayers comprising the phosphatidylserine surrounding a hydrophobic domain (castor oil), with the ibuprofen dispersed within the castor oil. See FIG. 3. Castor oil was selected, in part, because ibuprofen was found to be substantially more soluble in castor oil than other oils. Crystal and geode cochleates containing 50, 10, 2, 0.4, or 0.1 mg/kg of ibuprofen were prepared. Male Sprague Dawley rats were treated orally (one hour prior to right hind footpad injection of carrageenan) with vehicle, a commercial preparation of ibuprofen or ibuprofen formulated in either crystal or geode cochleates and were evaluated for effects of treatment on inflammatory edema. Animals were sacrificed five hours after dosing with test articles (four hours after carrageenan injection). Efficacy evaluation was based on weight difference due to inflammation-induced swelling in injected (right) vs. uninjected (left) paws. In addition, gastric mucosa were evaluated for differences in incidence and severity of mucosal congestion and erosions.

Results:

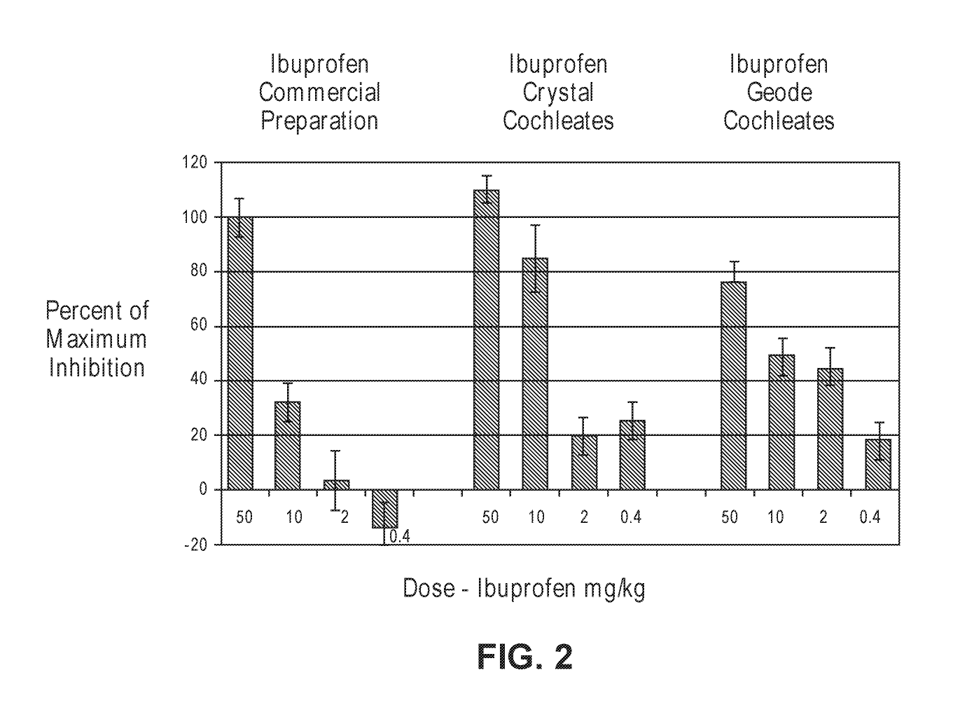

Treatment of rats with 50 mg/kg of all test articles or with 10 or 2 mg/kg ibuprofen geode cochleates or 10 mg/kg ibuprofen crystal cochleates resulted in significant inhibition of paw weight difference when compared to the vehicle control group. FIG. 2. The ibuprofen crystal cochleate formulation was most effective (ED50=6 mg/kg) followed by ibuprofen geode cochleates (ED50=7 mg/kg) and the commercial preparation of ibuprofen (ED50=30 mg/kg). Rats treated with 50 mg/kg of the commercial preparation of ibuprofen had significantly increased numbers of gastric lesions and increased total stomach lesion length. Some rats treated with 10 mg/kg the commercial preparation of ibuprofen also had gastric lesions. Rats given 50 mg/kg ibuprofen crystal cochleates had increased length and number of stomach lesions as compared to vehicle treated rats, but both parameters were significantly smaller when compared to the commercial preparation of ibuprofen treatment alone. Rats treated with 50, 10 or 2 mg/kg ibuprofen geode cochleates (ED50=7 mg/kg) had significant inhibition of carrageenan-induced paw edema with no evidence of gastric irritation. Surprisingly, the rats treated with 50 mg/kg of geode cochleates had no evidence of gastric lesions. Even though it was expected that the cochleate formulations might reduce the number of gastric lesions, it was not expected that such formulations could eliminate the gastric lesions altogether, as observed with the ibuprofen geode cochleates. The toxicity data for rats treated with ibuprofen at 50 mg/kg are summarized in the following table:

| | Number of | | |
|---|---|---|---|
| | Animals | Number of Lesions | Average Size of |
| | with Lesions | per Animal | Lesion |
| Commercial | 9 | 4.1 ± 0.8 | 7.2 ± 1.9 |
| ibuprofen | | | |
| ibuprofen crystal | 6 | 1.4 ± 0.5 | 1.6 ± 0.6 |
| cochleates | | | |
| ibuprofen geode | 0 | 0 | 0 |
| cochleates | | | |

Treatment with crystal cochleates at 10 mg/kg resulted in the greatest inhibition of paw edema for any formulation at this dose (FIG. 2) and there was no evidence of gastric irritation. There were no gastric lesions in rats given 2, 0.4 or 0.1 mg/kg of any formulation.

Conclusions:

Results of this study demonstrated that both ibuprofen cochleate formulations had substantially greater beneficial in vivo effects on carrageenan paw edema than the commercial preparation of ibuprofen, as measured by ED50 and summarized in the table below:

| | ED50 |
|---|---|
| Commercial | 30 mg/kg |
| ibuprofen | |
| ibuprofen crystal | 6 mg/kg |
| cochleates | |
| ibuprofen geode | 7 mg/kg |
| cochleates | |

Significant gastric lesions occurred in rats treated with single-dose 50 mg/kg (9 of 10) and to a lesser extent in rat treated with 10 mg/kg (1 of 10) ibuprofen, whereas, unexpectedly, no gastric lesions were observed in rats given 50 mg/kg of the ibuprofen geode cochleates (or any of the lower doses). Only 6 of 10 rats given 50 mg/kg the crystal cochleates and none treated with 10 mg/kg had gastric lesions. Therefore both efficacy as well as incidence and severity of gastric irritation were significantly improved by cochleate formulations of ibuprofen.

Those skilled in the art will recognize, or be able to ascertain using no more than routine experimentation, many equivalents to the specific embodiments of the invention described herein. Such equivalents are intended to be encompassed by the following claims.

1. A method of treating a subject in need thereof with an encochleated NSAID, wherein the subject has poor tolerability to a non-encochleated NSAID or is susceptible to NSAID-induced lesions or ulcers in the gastrointestinal tract, the method comprising orally administering to the subject a formulation comprising a cochleate, wherein the cochleate comprises a therapeutically effective amount of a NSAID.

2. A method of treating a subject in need thereof with an encochleated NSAID, the method comprising orally administering to the subject a formulation comprising a cochleate, wherein the cochleate comprises a therapeutically effective amount of a NSAID, and wherein the cochleate is a geode cochleate comprising:
a lipid monolayer comprising a negatively charged phospholipid, wherein the lipid monolayer surrounds a droplet of castor oil and the NSAID is comprised within the droplet of castor oil; and
a lipid strata comprising alternating divalent cations and phospholipid bilayers comprising the negatively charged phospholipid, wherein the lipid strata is disposed about the lipid monolayer.

3. The method of claim 1, wherein the cochleate is a geode cochleate comprising:
a lipid monolayer comprising a negatively charged phospholipid, wherein the lipid monolayer surrounds a hydrophobic domain and the NSAID is dispersed within the hydrophobic domain; and
a lipid strata comprising alternating divalent cations and phospholipid bilayers comprising the negatively charged phospholipid, wherein the lipid monolayer is sequestered within the lipid strata.

4. The method of claim 3, wherein the hydrophobic domain is an oil.

5. The method of claim 4, wherein the oil is castor oil.

6. The method of claim 1, wherein the NSAID is selected from the group consisting of a salicylate, a propionic acid derivative, an acetic acid derivative, an enolic acid derivative, an anthranilic acid derivative, a selective COX-2 inhibitor, a sulfonanilide, clonixin, licofelone and H-harpagide).

7. The method of claim 1, wherein the NSAID is a propionic acid derivative.

8. The method of claim 1, wherein the NSAID is an acetic acid derivative.

9. The method of claim 1, wherein the NSAID is an enolic acid derivative.

10. The method of claim 1, wherein the NSAID is a selective COX-2 inhibitor.

11. The method of claim 1, wherein the cochleate comprising the therapeutically effective amount of the NSAID does not induce any lesions in the gastrointestinal tract when administered to the subject.

12. The method of claim 1, wherein the geode cochleate reduces the proportion of subjects developing lesions or ulcers in the gastrointestinal tract of a subject by over 40%, 50%, 60%, 70%, 80%, 90%, or 100% as compared to the proportion of subjects developing lesions or ulcers in the gastrointestinal tract when treated with a nonencochleated version of the NSAID.

13. The method of claim 1, wherein the geode cochleate reduces the average number of lesions or ulcers in the gastrointestinal tract of a subject by more than 60%, 70%, 80%, 90%, or 100% as compared to a nonencochleated version of the NSAID.

14. The method of claim 1, wherein the geode cochleate reduces the average size of lesions or ulcers in the gastrointestinal tract of a subject by more than 50%, 60%, 70%, 80%, 90%, or 100% as compared to a nonencochleated version of the NSAID.

15. The method of claim 1, wherein the NSAID dose in the geode cochleate is reduced by more than 20%, 30%, 40%, 50%, 60% 70%, 80%, or 90% as compared to the dose of a nonencochleated version of the same NSAID.

16. The method of claim 1, wherein the subject has poor tolerability to a non-encochleated NSAID.

17. The method of claim 1, wherein the subject is susceptible to NSAID-induced lesions or ulcers in the gastrointestinal tract.

18. The method of claim 2, wherein the ratio between the hydrophobic domain (HD) and the phospholipid component of the geode cochleate (PPLGD) HD:PPLGD or the castor oil domain (COD) and phospholipid component of the geode cochleate (PPLGD) COD:PPLGD is 1:20 or less, 1:15 or less, 1:10 or less, 1:8 or less, 1:6 or less, 1:5 or less, 1:4 or less, 1:3.5 or less, 1:3 or less, 1:2.75 or less, 1:2.5 or less, 1:2.25 or less, 1:2 or less, 1:1.75 or less, 1:1.5 or less, 1:1.25 or lesser 1:1 or less.

19. The method of claim 1, wherein the subject is administered no more than 3 g, 2.5 g, 2 g 1.5 g, 1.25 g, 1 g, 750 mg, 500 mg, 400 mg, 300 mg, 250 mg, 200 mg, 150 mg, 100 or 50 mg per day of the cochleate.

20. The method of claim 1, wherein the cochleate is administered once a day or twice a day.

21. The method of claim 1, further comprising before the administering step, a step of identifying the subject as being susceptible to NSAID-induced lesions or ulcers in the gastrointestinal tract.

22. The method of claim 1, wherein the formulation further comprises bile salts.

23. The method of claim 22, wherein the cochleate formulation contains 0.1 mM to 0.5 mM bile salts.

24. The method of claim 1, wherein the subject is a mammal.

25. The method of claim 1, wherein the subject is a human.

26. The method of claim 1, wherein the cochleate comprises one or more negatively charged lipids, wherein the one or more negatively charged lipids comprise between 40% to 70% of the total lipid in the cochleate.

27. The method of claim 26, wherein the one or more negatively charged lipids comprise between 50% to 60% of the total lipid in the cochleate.

28. The method of claim 26, wherein the one or more negatively charged lipids comprise phosphatidylserine.

29. The method of claim 28, wherein the phosphatidylserine is soy phosphatidylserine.

30. The method of claim 1, wherein the cochleate further comprise one or more neutral or cationic lipid or sterols.

31. The method of claim 30, wherein the one or more neutral or cationic lipid or sterols are selected from the group consisting of phosphatidylcholine and sphingomyelin.
